(19) United States
(12) Patent Application Publication
(10) Pub. No.: US 2025/0073056 A1
(43) Pub. Date: Mar. 6, 2025

(54) MENSTRUAL RESERVOIR WITH VALVULAR OUTLET

(71) Applicant: Paul Robert Winegard, Gainesville, FL (US)

(72) Inventor: Paul Robert Winegard, Gainesville, FL (US)

(21) Appl. No.: 18/457,745

(22) Filed: Aug. 29, 2023

(51) Int. Cl. A61F 5/455; A61F 5/44

(52) U.S. Cl. CPC A61F 5/4553; A61F 5/4405

(57) ABSTRACT

A menstrual reservoir device with valvular outlet allowing for the collection and controlled draining of blood from the vaginal canal during the menstrual cycle. The device is intended be inserted into the vaginal canal where it will fix into place and collect menstruation. The reservoir comprises at least one outlet and the outlet will preferably extend towards or past the vaginal opening. The inventive device comprises a valvular portion to prevent fluid flowing from the reservoir when closed and allows fluid to flow when open. Embodiments comprise two general embodiments of reservoir cups and reservoir discs. In menstrual cup embodiments, the valvular portion serves to release suction force during device removal; when the valvular portion is open, air may enter the reservoir through the outlet, displace fluid and release the suction force of the reservoir allowing for easy removal.

### FIELD OF THE INVENTION

The present invention relates generally to reusable feminine hygiene products and their use in managing menstruation, and more specifically, to features that improve the insertion/removal process, improve sanitation, and improve overall comfort and ease of use during the removal and disposal of blood as well as reduce environmental waste.

### BACKGROUND OF THE INVENTION

Menstrual cups and discs are a relatively unadopted technology in the space of feminine hygiene and menstruation products when compared to other menstrual devices. Menstrual cup technology itself has been around since the 1930's and menstrual discs since the 1990's, and despite the cost saving advantage and environmental benefits of reusability, they have yet to receive widespread adoption. The reasons for this may include lack of education or habit in behavior, but there are also concerns related to ease of use and sanitation that may affect adoption. Traditional menstrual cups and discs must be inserted and removed through the insertion of the hands into the vaginal canal every time the device needs to be emptied. While there are differences between menstrual cups and discs, the primary mechanism of fixing the device into place in the vaginal canal to collect blood in a reservoir is common between them.

In the case of menstrual cups, the device is folded, inserted and allowed to expand, creating suction against the vaginal canal allowing the reservoir to collect menstrual blood without slipping. This suction comes at the cost of difficulty in removal. While many menstrual cups come with handles or grip at the bottom of the cup, most users of these cups will not be able to simply pull the cups out easily as the suction can make it difficult. Many menstrual cup users report pain or discomfort when pulling against the suction and instead reach into the vaginal canal to pinch the cup and break the suction before removing. The removal techniques of the cup often lead to unsanitary insertion of the hands and difficulty in extraction that may lead to mess in the spilling of menstrual blood from the cup. The main complaints of users tend to focus around discomfort, difficulty in removal, frequency of removal and reinsertion as well mess from spilling menstrual blood and other sanitation concerns.

In the case of menstrual discs, the device is inserted further into the vaginal canal than menstrual cups and rests near to the cervix. The elastically flexible but firm rim fits snugly against the vaginal fornix between the pubic bone and posterior fornix where the open mouth of the reservoir faces the cervix and collects blood. Menstrual discs do not create suction and thus do not share the discomfort of resistance provided by the suction force during extraction. The main complaints of menstrual discs generally relate to difficulty in insertion, mess and difficulty during extraction, as well as instability of the device during the menstrual cycle. As menstrual discs are positioned near the cervix, the device requires that the user insert their hands far deeper into the vaginal canal to insert and remove the device. The device is fixed in place by maneuvering it to fit snugly against the vaginal fornix which may prove difficult for users to reach during the removal process and often result in mess and unsanitary practices.

Many users of menstrual discs report that it is nearly impossible to remove a disc full of menstrual blood without spilling on the hands or elsewhere. Also, as the device must fit near to the cervix against the vaginal fornix, the reservoir portion of the device is disc shaped rather than cup shaped and is generally of a lower capacity than menstrual cup and disposable absorbent technologies, requiring that the user remove and empty the disc more often than they might like to.

The present invention attempts to solve the problems associated with both menstrual cups and discs as well as others.

### SUMMARY OF THE INVENTION

Provided herein are systems, methods and apparatuses for a Menstrual Reservoir with Valvular Outlet. In one embodiment, the Menstrual Reservoir with Valvular Outlet comprises a flexible and elastic reservoir herein referred to as “the reservoir portion” of the device. The reservoir portion has at least one outlet, herein referred to as “the reservoir outlet portion” of the device. The reservoir outlet portion may be but is not limited to a hole or tubular structure of any length, preferably leading to the vaginal opening. The flow of blood through the reservoir outlet portion is controlled by at least one valve, herein referred to as “the valvular portion” of the device. The valvular portion comprises at least one inlet or inlet portion, at least one valve mechanism and at least one outlet or outlet portion. When the the valvular portion is in a closed configuration, flow is arrested and the reservoir portion and reservoir outlet portion serve as a cavity to collect menstrual blood; when the valvular portion is in an open configuration, flow is allowed and the reservoir outlet portion serves to drain menstrual blood from the reservoir out of the vaginal canal through the valvular outlet portion. When the valvular portion is in an open configuration, air is allowed to flow into the reservoir through the outlet portion, both displacing the collected menstrual blood and after emptying the reservoir, releasing suction force if present.

The mechanism of the valvular portion may refer to any process either mechanical or electrical which may selectively arrest or allow the flow of fluid, this may comprise of but is not limited to the following: ball valves, pinch valves, clamp valves, twist control valves, shutter valves, push/pull valves, button valves, mechanical valvular mechanisms of any kind, solenoid valves, electrostatic valves, electrical/magnetic valves of any kind, removable end caps or removable/movable/switchable obstructions to the outlet portion of any kind, zippers, fasteners, interlocking sections, joints, plungers, syringes, pressurized tubes, extraction devices, or any other mechanism in which the reservoir may be selectively drained allowing for the removal of collected blood without the removal of the Menstrual Reservoir from the vaginal canal.

In various embodiments, the valvular portion may be permanently fixed to the reservoir outlet portion or it may be detachable, either for ease of use, ease of cleaning, ease of storage or for any other reason. The valvular portion of the device may function either through a mechanism that remains attached to the reservoir as it is collecting menstruation in the vaginal canal, or it may function as a device that is disconnected from the main reservoir until it is attached to the main reservoir when the reservoir is to be drained.

In one embodiment in which the reservoir portion is a menstrual cup, a method for using the Menstrual Reservoir with Valvular Outlet comprises: setting the valvular portion of the device to a closed configuration, folding the reservoir portion of the device to a folded configuration of diameter D3 from an unfolded configuration of diameter D1 and inserting the diameter D3 of the folded configuration into the vaginal canal towards the cervical opening; as the device is inserted, the flexible cup portion will expand to a partially folded configuration of diameter D2 and then return to a fully unfolded configuration of diameter D1; with a small amount of rotational adjustment, the rim of the reservoir portion will create a suction force sealing the device in the vaginal canal with the open mouth of the reservoir portion facing the cervix; the reservoir will then collect menstrual blood during the menstruation cycle until the reservoir is sufficiently full and in need of draining; the reservoir portion may then be drained by setting the valvular portion of the device to an open configuration; air will flow into the reservoir from the outlet portion and the blood will flow from the reservoir through the outlet portion and exit the vaginal canal. After blood has drained, the valvular portion may then be left in an open configuration and by pulling on the reservoir outlet or valvular portion, air is allowed to enter the reservoir and release the suction force allowing for easy extraction of the device. The device may then be cleaned, sanitized and the process repeated until menstruation has ceased. The device may be used in subsequent menstruation cycles in the same fashion.

Alternatively, in another method, after insertion and menstruation, once the reservoir is sufficiently full, the valvular portion may be opened and the reservoir only partially drained; the air entering the reservoir will only displace the blood expelled from the outlet and suction will be preserved; the valvular portion may then be closed and the device may be left inside the vaginal canal; the reservoir may then be drained partially as it refills with new menstruation in a repeated fashion until menstruation has ceased; the device may then be removed, cleaned and sanitized and reused in subsequent menstruation cycles.

In another embodiment in which the reservoir is a menstrual disc, a method of using the Menstrual Reservoir with Valvular Outlet comprises: setting the valvular portion of the device to a closed configuration, pinching the rim of the reservoir into a pinched configuration, inserting the pinched reservoir into the vagina as far back as can be achieved, flipping the reservoir and tucking the front rim of the reservoir behind the pubic bone in the vaginal fornix with the open mouth of the reservoir facing the cervix. After allowing menstruation to occur for a certain period of time, the reservoir may then be drained by setting the valvular portion of the device to an open configuration allowing blood to drain from the reservoir. As the device is held in place by fitting against the vaginal fornix, there is no suction force that can be released, so the user may allow the reservoir to drain fully without worry of the device falling out of place. The user may then set the valvular portion to a closed configuration and repeat the cycle of allowing menstruation to collect before draining. Once menstruation has ceased, the end user may then set the valve to an open configuration and remove the device either by reaching in to grab the reservoir, or by pulling on the valvular portion or reservoir outlet portion, causing flexion of the reservoir rim and extraction of the device.

The methods, systems and apparatuses are set forth in part in the description which follows, and in part will be obvious from the description or can be learned by practice of the methods, systems and apparatuses. The advantages of the methods, systems and apparatuses will be realized by means by the means of the elements and combinations particularly pointed out in the appended claims. It is to be understood, both the forgoing general description and the following detailed description are exemplary and explanatory only and are not restrictive of the methods systems and apparatuses, as claimed.

### BRIEF DESCRIPTION OF THE DRAWINGS

In the accompanying figures, like figures are accompanied by like references numerals and like alphanumeric designations among the several preferred embodiments of the present invention.

FIG. 1 is a front view of a the Menstrual Reservoir with Valvular Outlet, according to one embodiment. In this embodiment, the valvular mechanism is a mechanical, hand operated ball valved fixed to the end of a length of tube serving as the reservoir outlet, and the reservoir is a menstrual cup.

FIG. 2 is a side view of the menstrual reservoir with valvular outlet according to the same embodiment as FIG. 1.

FIG. 3 is a top perspective view of the menstrual reservoir with valvular outlet, according to the same embodiment as FIG. 1 and FIG. 2.

FIG. 4 is a graphic demonstrating the process of folding the reservoir in preparation for insertion into the vaginal canal according to one embodiment in which the reservoir is a menstrual cup.

FIG. 5 is a bottom view of the menstrual reservoir with valvular outlet according to one embodiment in which the valvular portion is in a closed configuration. The mechanism is a rotational handle controlling a ball valve and the reservoir is a menstrual cup.

FIG. 6 is a bottom view of the menstrual reservoir with valvular outlet according the same embodiment as FIG. 5 in which the valvular portion is in an open configuration. The mechanism is a rotational handle controlling a ball valve and the reservoir is a menstrual cup.

FIG. 7 is a graphic demonstrating the collection of menstrual blood in the menstrual reservoir when the valvular portion is in a closed configuration and the reservoir cup is fixed in place in the vaginal canal according to one embodiment in which the reservoir is a menstrual cup; the reservoir is in a “full” state.

FIG. 8 is a graphic demonstrating the drainage of menstrual blood from the menstrual reservoir when the valvular portion is in an open configuration and the reservoir cup is fixed in place in the vaginal canal according to one embodiment in which the reservoir is a menstrual cup; the reservoir is in a “draining” state.

FIG. 9 is a front view of the menstrual reservoir with valvular outlet, according to one embodiment in which the reservoir is a menstrual cup. In this embodiment, the valvular mechanism is a mechanical, hand operated twist control tube end valve fixed to the end of a length of tube serving as the reservoir outlet.

FIG. 10 is a front view of the menstrual reservoir with valvular outlet, according to one embodiment in which the reservoir is a menstrual cup. In this embodiment, the length of tube serving as the reservoir outlet is much longer than embodiments illustrated in the previous figures. Fixed to the end of the reservoir outlet tube is a twist operated valvular mechanism identical to the mechanism in FIG. 9.

FIG. 11 is a 6 step diagram displaying the process for inserting a menstrual cup embodiment of the Menstrual Reservoir with Valvular Outlet.

FIG. 12 is a front view of a menstrual cup embodiment of the Menstrual Reservoir with Valvular Outlet in which the main reservoir body is in a pinched configuration.

FIG. 13 is a top perspective view of a menstrual cup embodiment of the Menstrual Reservoir with Valvular Outlet in which the main reservoir body is in a pinched configuration.

FIG. 14 is a graphic displaying the extraction of a menstrual cup embodiment of the Menstrual Reservoir with Valvular Outlet in which the valvular portion of the device is in an open configuration and the reservoir portion is being pinched by the vaginal canal as it is extracted; as it is extracted, air flows through the valvular portion to release suction forces in the reservoir portion.

FIG. 15 is a front view of a menstrual disc embodiment of the Menstrual Reservoir with Valvular Outlet.

FIG. 16 is a top perspective view of a menstrual disc embodiment of the Menstrual Reservoir with Valvular Outlet.

FIG. 17 is a top perspective view of a menstrual disc embodiment of the Menstrual Reservoir with Valvular Outlet in which the rim and main body of the reservoir are in a pinched configuration.

FIG. 18 is a side perspective view of a menstrual disc embodiment of the Menstrual Reservoir with Valvular Outlet in which the rim and main body of the reservoir are in a pinched configuration and rotated to be horizontal with the reservoir outlet channel.

FIG. 19 is a graphic displaying the insertion of a menstrual disc embodiment of the Menstrual Reservoir with Valvular Outlet; the disc is being tucked behind the pubic bone in the vaginal fornix.

FIG. 20 is a graphic demonstrating the collection of menstrual blood in the menstrual reservoir when the valvular portion is in a closed configuration and the reservoir cup is fixed in place in the vaginal canal according to one embodiment in which the reservoir is a menstrual disc; the reservoir is in a “full” state.

FIG. 21 is a graphic demonstrating the drainage of menstrual blood from the menstrual reservoir when the valvular portion is in an open configuration and the reservoir cup is fixed in place in the vaginal canal according to one embodiment in which the reservoir is a menstrual disc; the reservoir is in a “draining” state.

FIG. 22 is a graphic demonstrating the removal of a menstrual disc embodiment of the Menstrual Reservoir with Valvular Outlet; the rim of the disc reservoir is being flexed out of the vaginal fornix due to the reservoir outlet portion being pulled down out of the vaginal opening.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with references to the accompanying figures, wherein like numerals and like alphanumeric designations reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises”, “comprising”, “includes”, and/or “including” when used herein, specify the presence of stated features, integers, steps, operations, elements and/or components, but do not preclude the presence or addition of one or more other features, integers, steps, operations, elements, components, and/or groups thereof. Recitation of ranges of values herein are merely intended to serve as a shorthand method of referring individually to each separate value falling within the range, unless otherwise indicated herein, and each separate value is incorporated into the specification as if it were individually recited herein. The word “about”, when accompanying a numerical value, is to be construed as indicating a deviation of up to and inclusive of 10% from the stated numerical value. The use of any and all examples, or exemplary language (“e.g.” or “such as”) provided herein, is intended merely to better illustrate the invention and does not pose a limitation on the scope of the invention unless otherwise claimed. No language in the specification should be construed as indicating any non claimed element as essential to the practice of the invention.

References to “one embodiment”, “an embodiment”, “example embodiment”, “various embodiments”, etc., may indicate that the embodiment(s) of the invention so described may include a particular feature, structure or characteristic, but not every embodiment necessarily includes the particular feature, structure or characteristic. Further, repeated use of the phrase “in one embodiment”, or “in an exemplary embodiment” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Generally speaking, the Menstrual Reservoir with Valvular Outlet is a reusable feminine hygiene product and the method of using the Menstrual Reservoir with Valvular Outlet is for managing menstruation, and more specifically, to features that improve/speed up the insertion and removal process, improve/speed up the process of disposing blood, improve capacity/wear time, reduce mess/cleanup, reduce unsanitary hygiene practices, improve the period experience in general and reduce environmental waste.

The Menstrual Reservoir with Valvular Outlet is a simple, reusable alternative to menstrual pads, sanitary napkins, tampons, and disposable undergarments as well as preexisting reusable menstrual devices, such as menstrual cups, discs or washable undergarments. The Menstrual Reservoir with Valvular Outlet may in some ways be considered as an intended improvement to existing menstrual cup, menstrual disc and other menstrual reservoir technologies in market today.

The Menstrual Reservoir with Valvular Outlet comprises a reservoir portion facing towards the cervical opening. The reservoir portion may vary significantly in various embodiments and may be fixed in place similar to preexisting menstrual cups or preexisting menstrual discs. According to various embodiments, the reservoir is flexible and provides a method for easy insertion and may be able to create suction in the vaginal canal upon expansion or it may be fixed in place through resting against the vaginal fornix near the cervix. After fixed in place, the reservoir serves to collect the flow of menstruation exiting from the cervix into the vaginal canal. The reservoir cup comprises a diameter and rim height specifically selected based on the anatomical features of the female body. Pelvic floor strength (influenced by age, childbirth, weight and muscularity among other factors), cervix height (accounting for both baseline cervix height and changes that may occur during the menstrual cycle) and fluid capacity are all size, property and dimension considerations for the Menstrual Reservoir with Valvular Outlet.

The various embodiments of the Menstrual Reservoir with Valvular Outlet will fall into two main categories: Menstrual Cup embodiments and Menstrual Disc embodiments; what differs between these embodiments is the method in which the device is fixed into place in the vaginal canal: in the case of menstrual cup embodiments, the device is fixed into place through a suction force in the vaginal canal caused through reservoir expansion, in the case of menstrual disc embodiments, the device is fixed in place by fitting against the vaginal fornix between the pubic bone and posterior fornix. What is common between all embodiments is a reservoir portion facing the cervical opening designed to catch menstrual fluid exiting the cervix and a method for fixing the device in the vaginal canal.

The following description and accompanying figures will focus on the Menstrual Cup embodiment category of the Menstrual Reservoir with Valvular Outlet followed by a shorter description of a Menstrual Disc embodiment of the device with some accompanying figures. There are far more similarities than differences between the two embodiment categories, as the main distinction between the two is related to insertion/removal, as well as difference in the forces holding the device in place. The novelty and improvement in mechanism provided by the Menstrual Reservoir with Valvular Outlet is common between Menstrual Cup embodiments and Menstrual Disc embodiments and differs only in the effect it has on the suction force of Menstrual cup embodiments. The primary improvements offered by the Menstrual Cup with Valvular Outlet are in the ease of controlled drainage of fluid from the reservoir, a substantial increase of wear time without need of extraction, as well enabling extraction of the device without needing to insert hands into the vaginal canal. These improvements are common between both menstrual cup and menstrual disc embodiments.

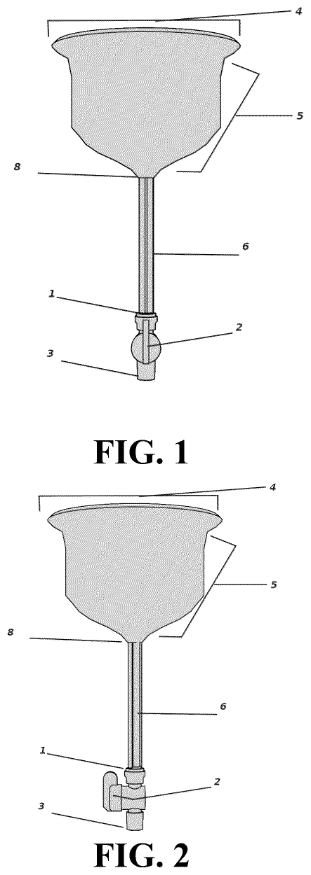

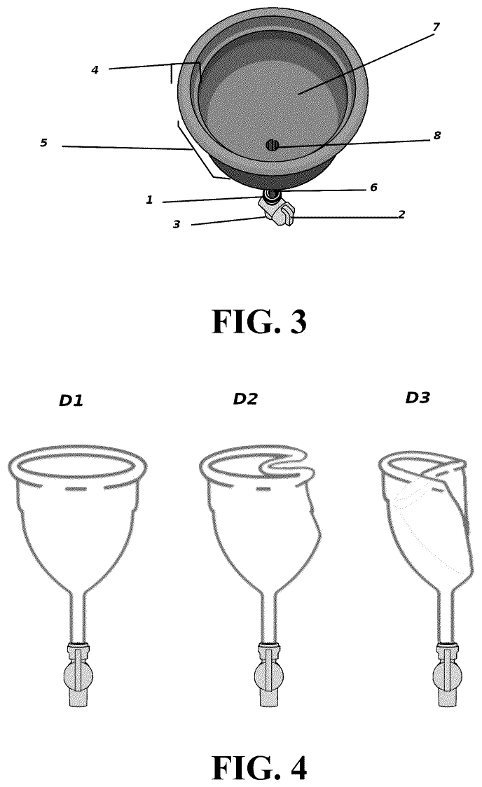

The material selected for the reservoir portion may vary between very flexible/elastic, flexible/elastic, somewhat flexible/elastic, somewhat rigid/elastic and rigid/elastic, as long as the device is able to be inserted comfortably and the device may be able to be fixed in the vaginal canal, the material flexibility of the reservoir may vary significantly. The reservoir portion comprises a rim near the opening of the reservoir cup nearest to the cervix. This rim may be a similar diameter as the main body of the reservoir cup, but it is preferably of a slightly larger diameter to assist in creating a seal for the suction force against the vaginal canal. As shown in FIG. 1, FIG. 2 and FIG. 3, in this embodiment, the reservoir rim 4 is of a larger diameter than the external main body 5 of the reservoir portion. The rim of the reservoir portion may be made of a material identical to the main body of the reservoir portion, but it also be made of a material comprising different properties such as flexibility/rigidity, elasticity, adhesiveness, etc. The selection of this and other materials will generally be tailored according to consumer preference and constraints of individual female anatomies as well as convenience in manufacturing, durability and aesthetic concerns among others.

As show in FIG. 3, the external main reservoir body 5 is of a larger diameter than the internal main reservoir body 7. The difference between the diameters of the external main reservoir body 5 and internal main reservoir body 7 denotes the thickness of the material used to construct the main reservoir body. The internal main reservoir body 7 comprises empty space for the collection of menstrual fluid. The variation in diameter, height and curvature of the external main reservoir body 5 and internal main reservoir body 7 contribute to the amount of fluid that may be collected during menstruation before drainage is required. As shown in FIG. 4, the main body of the reservoir in menstrual cup embodiments is generally flexible and may be folded, decreasing the diameter from unfolded diameter D1 to partially folded diameter D2 and finally to fully folded diameter D3. After folding, folded diameter D3 will be significantly smaller than the unfolded diameter D1.

The reservoir portion comprises a reservoir outlet portion allowing for the drainage of fluid from the main body of the reservoir. The reservoir outlet portion comprises at least one opening of the main reservoir body. In one embodiment, as shown in FIG. 3, the entrance to the reservoir outlet portion 8 is positioned at the bottom of the reservoir cavity. As shown in FIG. 1 and FIG. 2, the entrance to the reservoir outlet portion 8 leads directly into a reservoir outlet tube 6. The reservoir outlet tube 6, the entrance to the reservoir outlet portion 8, the main body of the reservoir, both external 5 and internal 7 as well as the reservoir rim 4, may all be collectively considered as the reservoir portion as they all assist in the collection of blood. The internal empty space of the reservoir rim 4, the internal main body 7 and the internal space of the reservoir outlet entrance 8 and reservoir outlet tube 6 may all serve to collect and hold menstrual fluid.

The reservoir outlet entrance 8 and reservoir outlet tube 6 may both be considered collectively as the primary components of the reservoir outlet. The reservoir outlet may be made of a material identical to the main reservoir body, but it may also be made of a material comprising different properties of rigidity, flexibility, elasticity, or any other aesthetic or material quality. The reservoir outlet does not have the same constraints of flexibility/elasticity as the main reservoir body, and may range from very flexible to fully rigid. The relative flexibility/elasticity of the main body and rim of the reservoir is necessary to provide the mechanism of creating suction in the vaginal canal. The reservoir outlet does not necessarily need to contribute to the creation of suction through the displacement of air, and may be considered as primarily and inlet for air into the reservoir and outlet for fluid from the reservoir. The material properties of the reservoir outlet may vary significantly in various embodiments, but common to all embodiments, the reservoir outlet must allow for the drainage of fluid from the main reservoir body. The reservoir outlet may be fixed to the main reservoir body by any number of means or processes and may vary significantly between different embodiments, but common to all embodiments, the reservoir outlet will comprise an air tight seal in which the main reservoir body and reservoir outlet may be considered as fused and both considered as primary components of the reservoir.

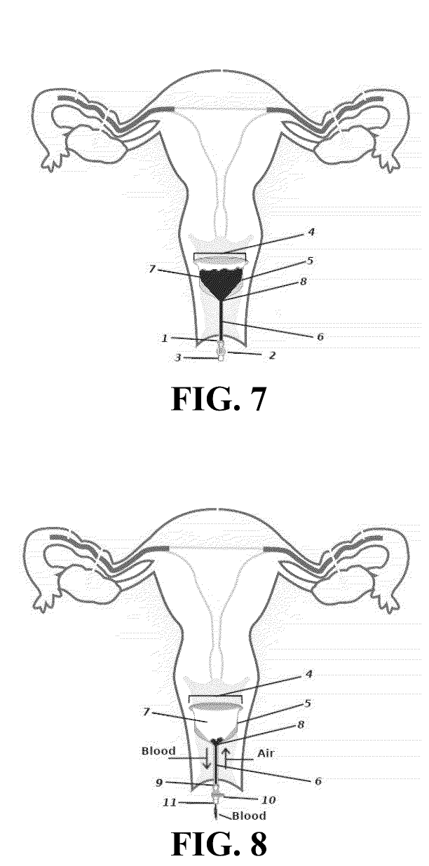

Embodiments of reservoir outlet portions may be permanently fixed to the main reservoir body, or may be able to be detached/removed from the main reservoir body either to assist in cleaning/sanitation, make the device more compact for storage or any other reason. The reservoir outlet may comprise any length of material and vary from an opening without length to a long reservoir outlet. FIG. 1, FIG. 2 are representations of one embodiment with moderately long reservoir outlet 6 in which the end may only protrude slightly from the vaginal canal as illustrated in FIG. 7 and FIG. 8. FIG. 10 is a representation of an embodiment in which the reservoir outlet portion 6 is significantly longer than the previously mentioned embodiment, and would extended far outside the vaginal opening. The length of the reservoir outlet can vary significantly and would be based on consumer preference. The reservoir outlet portion will preferably be long enough that the reservoir outlet extends external to the vaginal canal or just prior to the vaginal opening, but this is not required.

The reservoir outlet portion is controlled by a valvular portion. The valvular portion comprises an inlet portion, mechanism and outlet portion. The valvular portion serves to selectively arrest and allow the flow of liquid according to manually controlled or automated mechanism. The valvular portion may be located anywhere along the reservoir outlet portion or even extend into the reservoir body itself. The valvular portion may comprise any number of mechanisms, both mechanical and electrical as long as it allows selective control of fluid drainage from the reservoir outlet. The valvular portion can be set to an “open” state or “closed” state; when in an “open” state, the valvular portion allows fluid to flow through the reservoir outlet; when in a “closed” state, the valvular portion prevents fluid from flowing through the reservoir outlet.

As shown in FIG. 1, in this embodiment, a rotational ball valve is shown in a front facing perspective; the valvular portion comprises the valve inlet 1, valve mechanism control 2 and valve outlet 3. FIG. 1 shows the valvular portion in a closed position. This particular valve mechanism control 2 is in a closed when rotated vertically to be aligned with the reservoir outlet tube 6. As shown in FIG. 2, in an identical embodiment to FIG. 1, the valve mechanism control 2 can be seen from a side view.

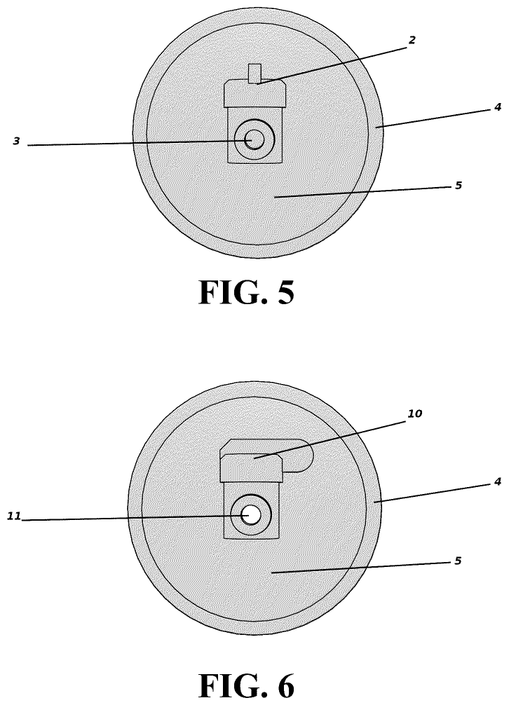

As shown in FIG. 5, in one embodiment, the device may be seen from a bottom view in which the valve mechanism 2 is in a closed configuration; the valvular outlet 3, can be seen blocking the reservoir outlet channel. As shown in FIG. 6, in the same embodiment as FIG. 5, the device may be seen from a bottom view in which the valve mechanism 10 is in an open configuration; the valvular outlet 11 can be seen to provide an opening to the reservoir outlet and an open channel through to the main reservoir body. The mechanism of the valvular portion may refer to any process either mechanical or electrical which may selectively arrest or allow the flow of fluid, this may comprise of but is not limited to the following: ball valves, pinch valves, clamp valves, twist control valves, shutter valves, push/pull valves, button valves, mechanical valvular mechanisms of any kind, solenoid valves, electrostatic valves, electrical/magnetic valves of any kind, removable end caps or removable/movable obstructions to the outlet portion of any kind, zippers, fasteners, interlocking sections, joints or any other mechanism in which the reservoir may be selectively opened allowing for the removal of collected blood.

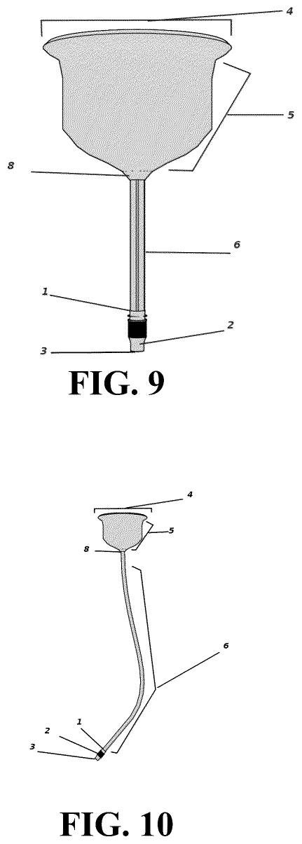

As shown in FIG. 1, FIG. 2, FIG. 3, FIG. 4, FIG. 5 and FIG. 6, the valvular mechanism selected for the primary illustration of an embodiment of the device is a rotational ball valve. This is in no way intended to convey preference towards ball valves, or rotational valve controls in the selection of mechanism. The rotational ball valve was selected for illustration purposes as it is easy and intuitive to see when it is in a “closed” state, as illustrated in FIG. 5 as valve mechanism 2, and when it is in a “open” state, as illustrated in FIG. 6, valve mechanism 10. As shown in FIG. 9 and FIG. 10, in a different embodiment, the mechanism 2 of the valvular portion is a twist valve similar to mechanisms that may be found on some urinary catheters. The selection of valvular mechanism is generally dependent on cost of manufacture, reliability of mechanism, ease of use, ease of cleaning/sanitation and comfort or preference of the end user. The valvular portion of the device comprises an inlet portion in which fluid is allowed to flow from the reservoir through the reservoir outlet portion when the valve mechanism is in an open configuration. In various embodiments, the inlet portion of the valvular portion may be fused permanently to the reservoir outlet or may be detachable to assist in cleaning/sanitation, repair or storage, what is common between all embodiments is that the inlet of the valvular portion makes an air-tight and fluid tight seal with the reservoir outlet portion.

As shown in FIG. 1, in one embodiment, the inlet of the ball valve 1 is connected to the reservoir outlet portion 6. As shown in FIG. 9, in another embodiment the inlet of the twist valve 1 is connected to the reservoir outlet portion 6. The valvular portion comprises an outlet. The outlet of the valvular portion may be of a similar size, shape and material to the inlet of the valvular portion, but this is not necessarily so. The valvular outlet may be of any shape, length and material, and may comprise multiple portions and materials. As shown in FIG. 5 and FIG. 6, in this embodiment, the valvular outlet 3/11 is simply extension of tubing connected to the valvular mechanism. As shown in FIG. 5, the valvular outlet 3 is in a closed configuration and leads to a blockage of the valvular mechanism 2 in a closed configuration. As shown in FIG. 6, the valvular outlet 11 leads to an open valvular mechanism 10 which in turn leads to the reservoir outlet portion and into the main reservoir body as one open channel. The valvular outlet serves as an outlet for menstrual fluid from the reservoir and vaginal canal, but may also serve as an inlet for air or additional fluid injected into the device. In various embodiments, the valvular outlet may connect to nothing or it may connect to tubular extensions or even other devices designed to create suction, inject fluid or any other number of materials or devices; what is common between all embodiments is that the valvular outlet comprises the ability to dispose fluid from the reservoir portion of the device.

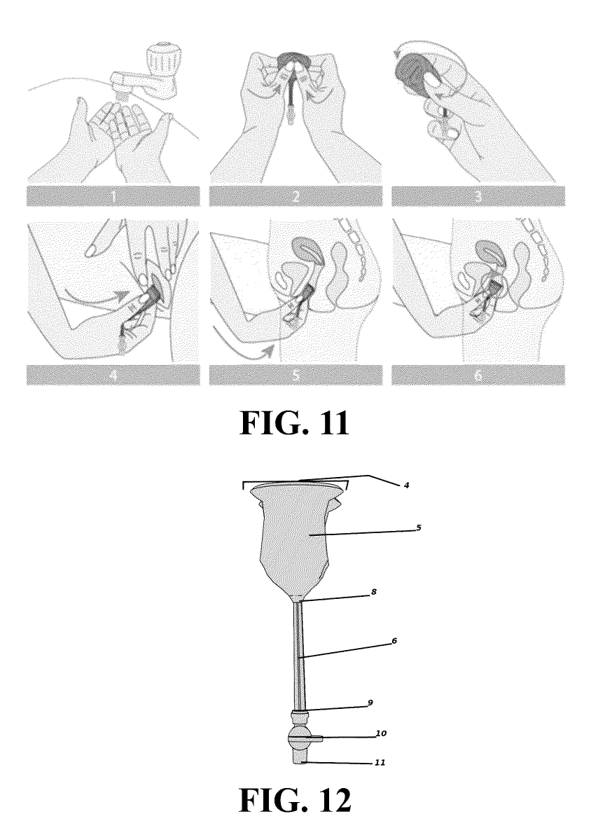

In one embodiment, a method for the use of the device is as follows: The user of the device either prior to menstruation has begun or when menstruation has begun will set the valvular portion of the device into a closed configuration as shown in FIG. 1, FIG. 2 and FIG. 5. This prevents air or fluid from flowing through the valvular portion and reservoir outlet portion of the device. As shown in FIG. 11, the user will insert the device into the vaginal canal as follows: As shown in FIG. 11 step 1, the user will clean and sanitize their hands and the device prior to insertion. As show in FIG. 11 step 2, the user will pinch the main reservoir body into a smaller, partially folded diameter; a partially folded state can also be seen shown in FIG. 4 labeled as D2. As shown in FIG. 11 step 3, the user will further fold the pinched main reservoir into a smaller, fully folded diameter; a fully folded state can also be seen shown in FIG. 4 labeled as D3. As shown in FIG. 11 step 4, the user will then proceed to insert the folded device into the vaginal opening with the rim of the reservoir portion entering first. As shown in FIG. 11 step 5, the user will push the reservoir body deeper into the vaginal canal, and as they do so, the main body of the reservoir will begin to transition from a fully folded state, as seen in FIG. 4 as D3 back towards a partially folded state, as seen in FIG. 4 as D2. As shown in FIG. 11 step 6, once the device has been inserted sufficiently into the vaginal canal, the user will then twist the device which will allow the main reservoir body to expand back to a fully unfolded state as seen in FIG. 4 as D1; when twisted, the rim of the device seen in FIG. 1 label as 4 will expand and create a seal against the vaginal wall. When the main reservoir body expands, the seal of the reservoir rim along with expanding body will create a vacuum suction force holding the device in place in the vaginal canal with the cup of the reservoir portion facing towards the cervix ready to collect menstrual blood.

The user will then be free to pursue other activities as their menstruation progresses for a given period of time. This period of time may vary upon different factors such as reservoir size or rate of flow. After a sufficient period of time has past, the reservoir will collect enough blood to be considered as in a “full” state, as shown in FIG. 7. The user will then proceed to position their vaginal opening, or depending on the specific embodiment, the end of the outlet portion in such a way that fluid released from the valvular outlet portion may be conveniently disposed (e.g. positioning the valvular outlet portion where it is facing a toilet bowl). The user will then set the valvular mechanism to an open configuration which will change the reservoir portion from a full state into a “draining” state, as seen in FIG. 8. As shown in FIG. 7, the valvular inlet 1 is arresting flow as the valvular mechanism 2 is in a closed state; the valvular outlet 3 has no flow of fluid or air as a result. As shown in FIG. 8, the valvular inlet 9 is allowing flow as the valvular mechanism 10 is in an open state; the valvular outlet 11 is expelling blood; blood flows from the internal reservoir 7 out of the reservoir outlet entrance 8 through the reservoir outlet tube 6 through the valvular inlet 9 past the open valvular mechanism 10 and finally out of the valvular outlet 11. As shown in FIG. 8, as blood is expelled from the reservoir out of the valvular portion, simultaneously, air is allowed to flow in through the valvular outlet 11 and up through the reservoir outlet portion 6 and finally into the internal main reservoir body 7 displacing blood as assisting in the expelling of fluid. Two potential methods of fluid drainage will now be described:

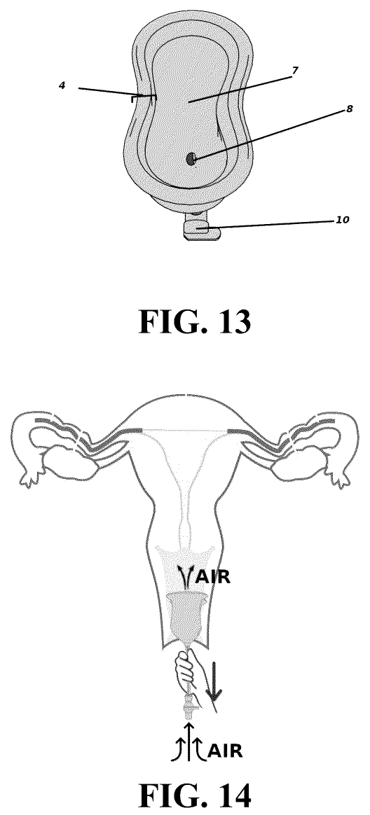

In one method, herein referred to as Method A, the user may allow the fluid to fully drain from the reservoir and reservoir outlet and the reservoir may be considered to be in an “empty” state. When the device is in an empty state, additional air that flows into the main reservoir body will no longer displace blood as there is no more blood left in the reservoir. The user may then pull on the valvular outlet portion of the device, and as the device is drawn against the narrowing vaginal canal, this will cause the device to enter a pinched configuration of diameter D2 as shown in FIG. 12 and FIG. 13. As shown in FIG. 14, as the user pulls, air will rush in through the open valvular outlet portion into the internal reservoir body and break the suction force against the vaginal canal; this will allow for easy extraction of the device as the user continues to pull the reservoir outlet portion until the device is fully extracted. The user may then clean the device and reinsert it, repeating the previous steps of the aforementioned method until menstruation has ceased and the device may be cleaned and stored for reuse in future menstruation cycles.

In another method, herein referred to as Method B, after the reservoir has entered a draining state, the user may elect to close the valvular mechanism prior to the reservoir entering an empty state. This will maintain the suction force keeping the reservoir in place as the total volume of air entering the reservoir from the valvular outlet will equal the displaced volume of blood draining from the valvular outlet. This method will allow the user to leave the device inserted and repeat the process of partially draining the reservoir until menstruation has ceased when the device may finally be removed in a fashion identical to Method A. So concludes the detailed description of various design embodiments and various methods of operation relating to a Menstrual Cup embodiment of the Menstrual Reservoir with Valvular Outlet.

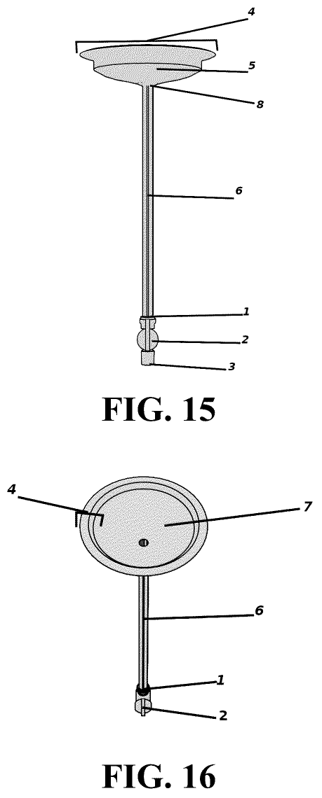

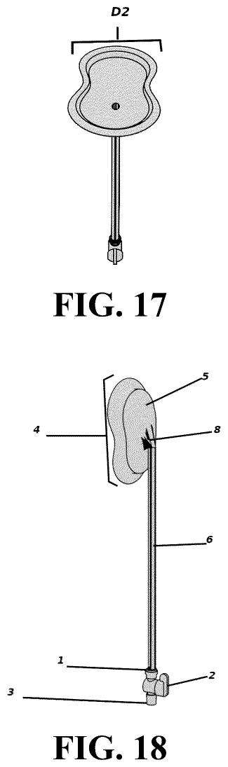

As aforementioned, the Menstrual Reservoir with Valvular Outlet may too have any number of Menstrual Disc embodiments. The primary difference between Menstrual Disc embodiments and Menstrual Cup embodiments is in the insertion/removal process, as well as the relationship the reservoir outlet portion and valvular portion have with suction forces in the reservoir portion. In Menstrual Disc embodiments, the reservoir is positioned much nearer to the cervix than in Menstrual Cup embodiments and is not held in place by a suction force, instead, in Menstrual Disc embodiments the reservoir is held in place by the rim of the reservoir fitting against the vaginal fornix between the pubic bone and posterior fornix. There is no air tight seal of the reservoir in Menstrual Disc embodiments as there are in Menstrual Cup embodiments. What is common between Menstrual Cup and Menstrual Disc embodiments is the function of the valvular mechanism in regards to the drainage of fluid from the reservoir portion through the reservoir outlet portion. As is shown in FIG. 15 and FIG. 16, in Menstrual Disc embodiments, the reservoir portion is more disc shaped than cup shaped. As is shown in FIG. 17 and FIG. 18, the rim 4 of the reservoir portion is elastically flexible, as is the main body 5 of the reservoir portion. In one embodiment, as shown in FIG. 18, the connection between reservoir cavity and reservoir outlet portion 8 is flexible allowing the disc to be rotated in such a way that the reservoir outlet portion 6 may lie flat against it. In a menstrual disc embodiment of the Menstrual Reservoir with Valvular outlets, a method for use is as follows:

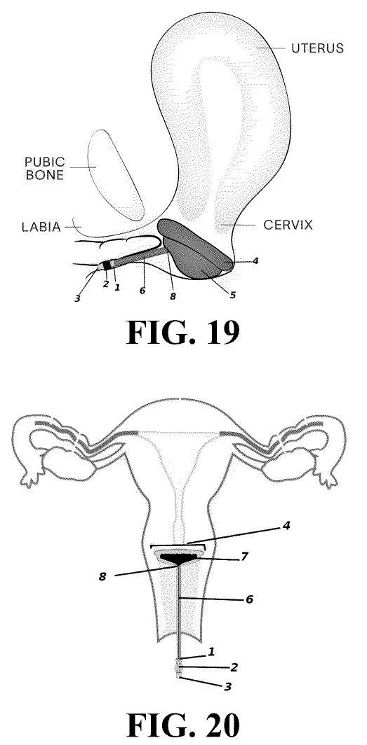

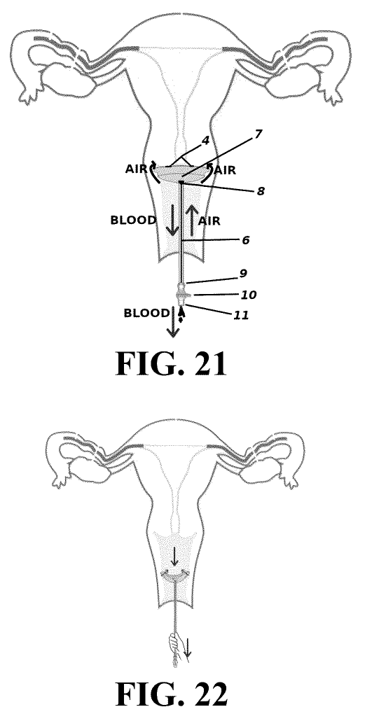

The device and hands are the end user are washed and sanitized. The valvular portion of the device is set to a closed configuration. As shown in FIG. 17, the rim of the reservoir is pinched to a smaller diameter of D2. As is shown in FIG. 18, the reservoir portion is rotated so that the cup lies horizontally parallel to the reservoir outlet channel 6. The device is then inserted cup side up into the vaginal canal. As shown in FIG. 19, the device is pushed back as far as it will go before being flipped and fit into place against the vaginal fornix, where the front of the rim is tucked behind the pubic bone and the reservoir outlet channel 6 leads downwards towards the vaginal opening. The device may then be operated in a method very similar to aforementioned methods A and B in menstrual cup embodiments of the device with a slight difference: in the case of menstrual disc embodiments, the reservoir may be fully drained without fear of the reservoir becoming dislodged, as there is no suction force which may be released. In one embodiment, as shown FIG. 20, the menstrual reservoir will have the rim of the reservoir 4 fixed against the vaginal fornix and collect blood. The blood will collect both in the internal reservoir body cavity 7 and the reservoir outlet channel 6. After the reservoir portion has entered a “full” state as shown in FIG. 20, the valvular portion of the device may then be set in an open configuration allowing the reservoir to begin drain as shown in FIG. 21. As shown in FIG. 21, there is no suction force which may be released when blood is being displaced from the reservoir so as blood exits the reservoir outlet channel 6, air is free to displace it both by entering through the reservoir outlet 6 and by entering the reservoir cavity from around the reservoir rim 4. The reservoir may be drained fully as the disc will be held in place by its position in the vaginal fornix as opposed to suction pressure, the valve may be set to a closed configuration, and the device left in place for the process to be repeated until menstruation has ceased, after which the device may be extracted by setting the valvular portion to an open configuration, and pulling on the valvular portion or reservoir outlet portion, causing the rim of the reservoir to flex and dislodge for the vaginal fornix and extract from the vaginal canal as shown in FIG. 22.

In both embodiment categories of menstrual cups and menstrual discs, the addition of a reservoir outlet and valvular mechanism greatly enhances the time in which the device can be left in the vaginal canal, greatly reduces the potential for mess in the spilling of menstrual blood, greatly improves sanitation by reducing the amount of time in which potentially unsanitary hands must be inserted into the vaginal canal, and greatly improves the ease and comfort of extracting the device from the vaginal canal. The innovation while differing slightly in menstrual disc and menstrual cup embodiments are of such similar nature they may both be considered as benefiting from the same innovation, namely the Menstrual Reservoir with Valvular Outlet.

The methods, drawings, diagrams, embodiments and examples mentioned in this detailed description are not meant to be interpreted as restricting or as limited to the inventive device or its intended use, but are merely intended to be illustrative of the device and potential methods of operation.

I claim:

1. A menstrual reservoir with valvular outlet comprising:
a) A reservoir portion in the vaginal canal allowing for the collection of blood during menstruation; the reservoir portion comprising a rim, and outer wall and a cavity allowing for the collection of blood in the vaginal canal;
b) the reservoir portion comprises at least one outlet allowing for fluid to be drained from the reservoir, referred to as the reservoir outlet portion;
c) the reservoir outlet portion will be connected to a valvular portion;
d) the valvular portion comprises at least one inlet, at least one valve mechanism and at least one outlet;
e) the valvular mechanism allows for the selective retention of fluid in the reservoir and controlled drainage of fluid from the reservoir through the reservoir outlet portion.

2. The menstrual reservoir with valvular outlet of claim 1, wherein the reservoir portion comprises a flexible and elastic rim, outer wall and cavity that will create a suction force in the vaginal canal fixing it in place.

3. A method of using the menstrual reservoir with valvular outlet of claim 2, wherein the valvular portion in conjunction with the reservoir outlet portion will allow for the selective drainage of menstrual fluid from the reservoir portion and vaginal canal without needing to remove the device from the vaginal canal; the method comprises:
a) closing the valvular portion of the device;
b) fixing the device in place, creating suction force in the vaginal canal;
c) allowing menstruation to occur, filling the reservoir;
d) setting the valvular portion to an open configuration;
e) partially draining the reservoir, preserving suction force;
f) setting the valvular portion to a closed configuration;
g) repeating the process of menstruation and partial draining of the reservoir until menstruation has ceased.

4. The menstrual reservoir with valvular outlet of claim 2, wherein the reservoir outlet portion in conjunction with the valvular portion extend to or out of the vaginal opening.

5. A method of extracting the menstrual reservoir with valvular outlet of claim 4 from the vaginal canal without needing to insert hands into the vaginal canal; the method comprises:
a) setting the valvular portion to an open configuration and draining any remaining fluid;
b) pulling on the valvular portion and/or reservoir outlet portion;
c) air will rush in through the valvular portion, breaking the suction force of the reservoir in the vaginal canal allowing the device to be extracted. The valvular portion or reservoir outlet portion can be pulled out without any resistance of suction, and the insertion of the hands into the vaginal canal to grab or pinch the reservoir or any attachments to the reservoir will not be necessary.

6. The menstrual reservoir with valvular outlet of claim 1, wherein the reservoir portion comprises a flexible and elastic rim, outer wall and cavity that will fit against the vaginal fornix between the pubic bone and posterior fornix fixing it into place.

7. The menstrual reservoir with valvular outlet of claim 6, wherein the valvular portion in conjunction with the reservoir outlet portion will allow for the selective drainage of menstrual fluid from the reservoir portion and vaginal canal without needing to remove the device from the vaginal canal.

8. The menstrual reservoir with valvular outlet of claim 6, wherein the reservoir outlet portion in conjunction with the valvular portion extend to or out of the vaginal opening.

9. A method of extracting the menstrual reservoir with valvular outlet of claim 8 from the vaginal canal without needing to insert hands into the vaginal canal; the method comprises:
a) setting the valvular portion to an open configuration and draining any remaining fluid;
b) pulling on the valvular portion and/or reservoir outlet portion;
c) the reservoir will flex and dislodge from the vaginal fornix and allow for extraction without the insertion of hands into the vaginal canal through pulling on the valvular portion and/or reservoir outlet portion.

10. The menstrual reservoir with valvular outlet of claim 2, wherein the device or any portion of the device is composed of any suitable elastomer, plastic, natural, or metal material or any combination of suitable materials.

11. The menstrual reservoir with valvular outlet of claim 6, wherein the device or any portion of the device is composed of any suitable elastomer, plastic, natural, or metal material or any combination of suitable materials.
